(19) United States
(12) Patent Application Publication
(10) Pub. No.: US 2024/0423148 A1
(43) Pub. Date: Dec. 26, 2024

(54) Hybrid Seed Production Method

(71) Applicant: BASF SE, Ludwigshafen Am Rhein (DE)

(72) Inventors: Frank Michiels, Gent (BE); Kirk Johnson, Davis, CA (US); Nanyen Chou, Sugar Land, TX (US)

(73) Assignee: BASF SE, Ludwigshafen Am Rhein (DE)

(21) Appl. No.: 18/827,196

(22) Filed: Sep. 6, 2024

Related U.S. Application Data
(63) Continuation of application No. 14/892,702, filed on Nov. 20, 2015, now abandoned.
(60) Provisional application No. 61/830,679, filed on Jun. 4, 2013.

(51) Int. Cl. A01H 1/00; A01H 1/02

(52) U.S. Cl. CPC A01H 1/123; A01H 1/023

(57) ABSTRACT

Methods are provided for hybrid seed production using 3-phyletic crosses between female, maintainer and male, particularly restorer lines, wherein the trait is introduced in the female line only at the stage of basic seed production via crossing of the female line with a maintainer line containing the gene or genes encoding the trait in homozygous state.

### CROSS-REFERENCE TO RELATED APPLICATIONS

This application is a Continuation of and claims the benefit of priority to U.S. application Ser. No. 14/892,702 filed on Nov. 20, 2015, which is a National Stage Entry of PCT/EP2014/060658 filed on May 23, 2014, which claims priority to U.S. Provisional Application No. 61/830,679 filed on Jun. 4, 2013, the contents of which are herein incorporated by reference in their entireties.

### FIELD OF THE INVENTION

The present invention relates to improved and versatile production schemes for hybrid seeds and plants having increased vigor or other qualities following from the crossing of genetically different male and female plant lines, wherein the hybrid seeds and plants further comprise a trait of interest such as an herbicide tolerance gene. More particularly, the invention is directed at a production process for hybrid seeds using 3-phyletic crosses between female, maintainer and restorer lines, wherein the trait is introduced in the female line only at the stage of basic seed production via crossing of the female line with a maintainer line containing the gene or genes encoding the trait in homozygous state.

### BACKGROUND OF THE INVENTION

Hybridization of plants is recognized as an important process in agriculture for producing progeny plants having a combination of desirable traits of the parent plants. The resulting hybrid progeny plants often have the ability to outperform parents in different traits such as yield, adaptability to environmental changes and disease resistance. This biological phenomenon in which the hybrid of two genetically dissimilar parents shows increased vigor at least over the mid-parent value ([P1+P2]/2) is known as “heterosis” or “hybrid vigor”. Hybrid seeds furthermore are also uniform in stand, development and characteristics. Today, hybrid seed production is predominant in agriculture and home gardening and has been one of the main contributing factors to the dramatic rise in agricultural output during the last half of the 20th century. In the US, the commercial hybrid seed market was launched in the 1920s, with the first hybrid maize.

For a number of reasons, primarily related to the fact that most plants are capable of undergoing both self-pollination and cross-pollination, the controlled cross-pollination of plants without significant self-pollination, to produce a harvest of hybrid seeds, has been difficult to achieve on a commercial scale. Many crop plants produce male and female reproductive organs on the same plant, usually in close proximity to one another in the same flower, favoring self-pollination.

To produce hybrid seed economically, self-pollination must be reduced or eliminated. This can be conveniently achieved in the female parent by elimination of the male reproductive organs or their functionality of producing viable pollen. Such sterility can be produced by hand emasculation, chemical or environmental emasculation, or manipulation of genetic male sterility or self-incompatibility. In monoecious plants with separate staminate and carpellate flowers, such as corn, removal of the male parts (tassel) can be performed easily on large scale. Large-scale esmaculation of species with perfect flowers, however, is economically unfeasible. The major types of male-sterility which are currently used in hybrid seed production include cytoplasmic male sterility (CMS), environmentally induced male sterility such as photoperiod male sterility (PGMS) or thermogenic male sterility, gametocide induced male sterility and transgenic male sterility system.

To produce hybrid seed using induced male sterility, be it environmentally or gametocide-induced, only male and female parent plant lines are required. The conditional male sterility can be relieved and the female parent plant line can be reproduced and multiplied by self-pollination. This is referred to as a 2-line or biphyletic hybrid seed production system.

CMS based hybrid seed production, on the contrary, requires 3 parent lines (triphyletic system). In addition to the male sterile (female) line and the restorer (male) line, an additional line is needed to maintain and multiply the female line: the so-called maintainer line.

CMS systems are controlled by the interaction of cytoplasmic and nuclear genes. The presence of at least one sterility inducing genetic factor in the cytoplasm of a plant, together with the absence of nuclear gene(s) causing any type of fertility restoration (or the presence of homozygous recessive nuclear genes for fertility restoration) makes a plant male sterile (A-line). The presence of a dominant fertility restorer gene in the nucleus of a plant will allow such plant to restore fertility in a hybrid after crossing it with an A-line. This line is called the R-line or restorer line. Because cytoplasm is maternally inherited, crossing of a plant of the female A-line with a plant which does not harbor nuclear gene(s) for any type of fertility restoration and does not have the cytoplasmic sterility inducing genetic factor, will result in male-sterile offspring plants. If these latter plants are otherwise isogenic to the female A-line plants, they can be used to reproduce and multiply (i.e. maintain) the A-line and are referred to as maintainer lines or B-lines.

Thus, hybrid seed production requires at least two cycles: a first cycle for A line multiplication wherein the female A-line is crossed with the male B-line to produce more A-line seeds and plants, and a second cycle for hybrid seed production wherein the female A-line is cross-pollinated by the male R-line to produce male fertile hybrid seeds. For commercial production of hybrid seeds, the multiplication of sufficient A-line female seed and plants for large-scale hybrid seed production is a staggered process involving multiple cycles of cross-pollination between the A-line and B-line plants, at increasing scales to produce pre-basic seed and finally basic seed, the latter being used for the hybrid seed production.

To maintain a high degree of purity and hybridity in the commercially produced hybrid seeds it is imperative that the basic seed of the A-line is a pure as commercially feasible. There are indeed many potential sources that may lead to impurities in the basic seed of the A-line including B-line seeds mixed into the pre-basic increases of the A-line, genetic variants mixed into A-line increases from volunteers in the field, errant pollen from neighbouring fields out-crossing onto the A-line in the multiplication or admixture into the A-line seed from any source during the industrial production, cleaning and seed processing steps. Such forms of genetic variants are usually removed from the fields by hand rogueing. If the plant-lines are characterized by specific herbicide tolerances, such rogueing by hand can be replaced by applying specific herbicide regimes.

The identification of appropriate A, B and R-lines characterized by the presence of desired agronomic characteristics and good combinability leading to significant heterosis in the commercial hybrid seed is time consuming and costly process. In particular, the breeding and identification of a good female line poses quite some challenges.

With the increasing acceptance of transgenic traits (as well as the increasing occurrence of non-transgenic traits including human-induced allelic variants or molecular marker characterized native traits) and the increasing combination of such traits into one hybrid plant, the task of hybrid seed production becomes even more challenging.

Conversion of any given A-line (and B-line) to include traits of interest by repeated crossings is a labor-intensive and time-consuming process. At the same time, the versatility to produce different combinations of traits in the hybrids is reduced, since the female lines will always have fixed particular combinations.

One solution could be to include traits in the hybrid seeds via the R-line only. Since R-lines are male fertile, they can be converted into R-lines containing the traits of interest by repeated back-crossing, which is a process that is quicker and more easily managed than A-line conversion. However, the resulting hybrid seed population will be completely hemizygous for the genes or alleles determining the trait.

CN1187292 discloses a method for increasing heterosis of crops or plants, characterized by the use of non-selective herbicide and its resistance gene in the process of producing hybrid seeds of plants and crops to ensure the purity in the hybrid population and the yield-increasing action of heterosis, eliminating the influence of sterile plants and other unwanted plants on yield, during the application of biphyletic, triphyletic and chemically induced heterosis, increasing the utilization level of heterosis and reducing seed cost.

WO98/48612 discloses a method for producing herbicide-resistant rice hybridizing seeds, comprising transferring a herbicide-resistant gene into the restorer of tri- or bi-lines rice hybrids, enabling the restorer harboring the herbicide-resistance gene to exhibit herbicide-resistance properties, then hybridizing the resultant transgenic restorer with the sterile line of the tri- or bi-lines, yielding herbicide-resistant rice hybrids. Using this method, impure rice hybrid seeds may be used in the production of rice, and the pseudo-hybridizing plants can be eliminated properly.

U.S. Pat. No. 6,066,779 describes a method for integrating the resistance gene to a non-selective herbicide into male parents and spraying the herbicide onto the hybrid population resulting from mating with the male parent for securing hybrid purity to reduce the strict demand for complete male sterility.

The prior art thus remains deficient in providing a versatile system for conversion of A-lines to A-lines comprising traits of interest and ensuing production of hybrid seed with the desired combination of traits of interest, having the additional advantage, in the case of herbicide tolerance traits, of the possessing the possibility to improve the purity of the hybrid seeds by herbicide application during the hybrid seed production process.

These and other problems are solved as described hereinafter in the different embodiments, examples and claims.

### SUMMARY OF THE INVENTION

In one embodiment, the invention provides a method for producing hybrid seed comprising an trait of interest or a gene of interest or a QTL of interest (such genes, events or QTLS contributing to insect control or tolerance, herbicide tolerance, stress tolerance, yield increase, oil content increase, starch increase, drought tolerance, cold tolerance or fiber yield increase) using a male-sterile plant line or A-line; a male-sterile plant line comprising said trait of interest in heterozygous or hemizygous state or AGOI-line; an isogenic maintainer plant line or B-line; an isogenic maintainer plant line comprising said trait of interest in homozygous state or BGOI-line; and a male fertile line such as a pollinator line or restorer line (R-line) comprising said trait of interest in homozygous state; wherein said hybrid seed is produced by crossing of basic seed of the AGOI-line and the R-line and collecting the seeds produced on plants of the A-line, and wherein said basic seed of the AGOI-line has been produced by crossing pre-basic seed of the A-line with pre-basic seed of the BGOI-line and collecting the seeds produced on plants of the AGOI-line, and wherein said pre-basic seed of the A-line has been produced by crossing pre-basic seed of the A-line with pre-basic seed of the B-line and collecting seeds produced on plants of the A-line.

In a particular embodiment the invention provides a method for producing herbicide-tolerant hybrid seed using a male-sterile plant line or A-line; a male-sterile plant line comprising a herbicide tolerance gene in heterozygous or hemizygous state or AHT-line; an isogenic maintainer plant line or B-line; an isogenic maintainer plant line comprising a herbicide tolerance gene in homozygous state or BHT-line; and a male fertile line such as a pollinator or restorer line or R-line comprising said herbicide tolerance gene in homozygous state; wherein said hybrid seed is produced by crossing plants grown from basic seed of the AHT-line and plants of the R-line and collecting the seeds produced on plants of the A-line, and wherein said basic seed of the AHT-line has been produced by crossing plants grown from pre-basic seed of the A-line with plants of the BHT-line and collecting the seeds produced on plants of the A-line, and wherein said pre-basic seed of the A-line has been produced by crossing plants grown from pre-basic seed of the A-line with plants of the B-line and collecting seeds produced on plants of the A-line.

The herbicide tolerance gene may provide tolerance against a herbicide selected from the group of acetyl CoA carboxylase inhibitors, acetolactoate synthase inhibitors, glutamine synthetase inhibitors, 5-enoylpyruvyl-shikimate-3-phosphate inhibitors, photosynthesis II inhibitors, diterpene synthesis inhibitors, hydroxyphenylpyruvate dioxygenase inhibitors, protoporphorinogen oxidase inhibitors, photosystem I electron diverters, microtubule inhibitors, lipid synthesis inhibitors, long chain fatty acid inhibitors or synthetic auxins. The herbicide tolerance may be provided by a transgene or by a variant allele endogenous to said plant.

The plant lines may be plant lines of rice (Oryza sativa), wheat (Triticum aestivum), corn (Zea mays), cotton (Gossypium hirsutum or G. barbadense), soybean (Glycine max), sorghum (Sorghum bicolor), rapeseed (Brassica napus), mustard seed (Brassica juncea), barley (Hordeum vulgare), oat (Avena sativa), rye (Secale cereale), pearl millet (Pennisetum typhoides), alfalfa (Medicago sativa), tomato (Lycopersicon esculentum), sugar beet (Beta vulgaris), sunflower (Helianthus annuus), onion (Allium cepa), petunia (Petunia hybrida), carrot (Daucus carota), sorghum (Sorghum spp.), cabbage (Brassica oleracea), melons (Cucumis melo), watermelons (Citrillus lanatus) or cucumber (Cucumis sativus) amongst others.

### BRIEF DESCRIPTION OF THE DRAWINGS

The following Examples, not intended to limit the invention to specific embodiments described, may be understood in conjunction with the accompanying Figures, incorporated herein by reference.

FIG. 1: Schematic representation of the steps in basic seed production and hybrid (rice) seed production using a triphyletic system.

FIG. 2: Schematic representation of the conversion of a B-line (left panel) or R-line (right panel) to a B-line or R-line comprising the gene(s) or allele(s) of interest conferring the particular trait(s) of interest.

FIG. 3: Schematic representation of the versatile methods described herein to produce hybrid seeds comprising a GOI.

### DETAILED DESCRIPTION OF EMBODIMENTS OF THE INVENTION

The current invention is based on the realization that traits, including herbicide tolerance traits can be introduced in hybrid plants and seeds which are produced using 3-phyletic crosses between female, maintainer and male fertile pollinator or restorer lines, at a late stage in the commercial seed production, i.e. only just prior to the crossing of the plants grown from basic seed to produce the hybrid seed.

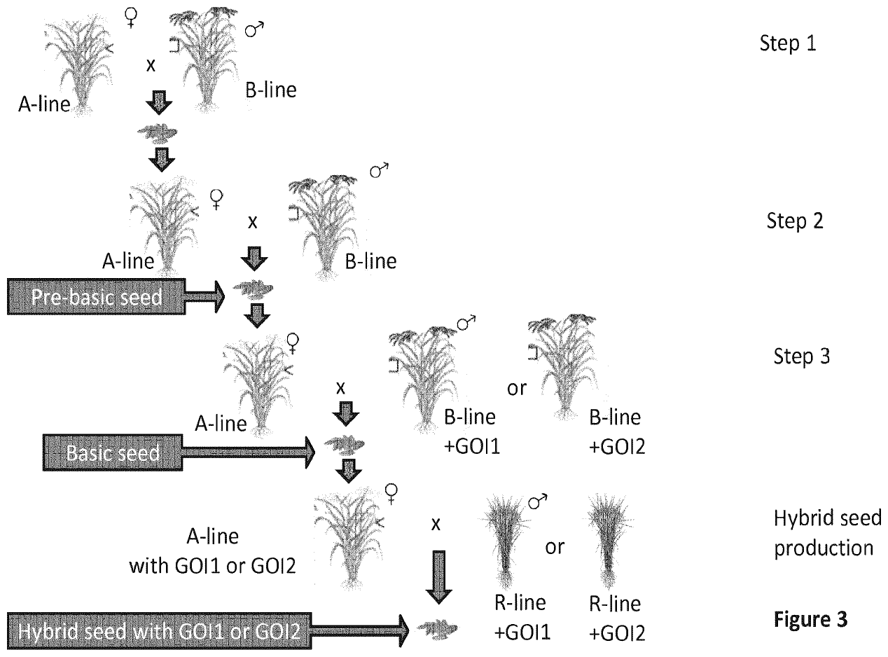

To this end, the trait is introduced in the female or A-line by crossing pre-basic seed of the A-line with an isogenic maintainer plant line or B-line which further comprises the gene(s) or allele(s) conferring the trait in homozygous state. The basic seed, collected from the female line now contains the gene(s) or allele(s) conferring the trait in hemizygous or heterozygous form. Upon growing plants from the basic seed and crossing with male restorer lines also containing the gene(s) or allele(s) conferring the trait in homozygous form, the hybrid seed will contain the gene(s) or allele(s) conferring the trait either in homozygous form (½ of the hybrid seed population) or in hemi- or heterozygous form (½ of the hybrid seed population) The method for producing hybrid seed containing the trait of interest is schematically represented in FIG. 3.

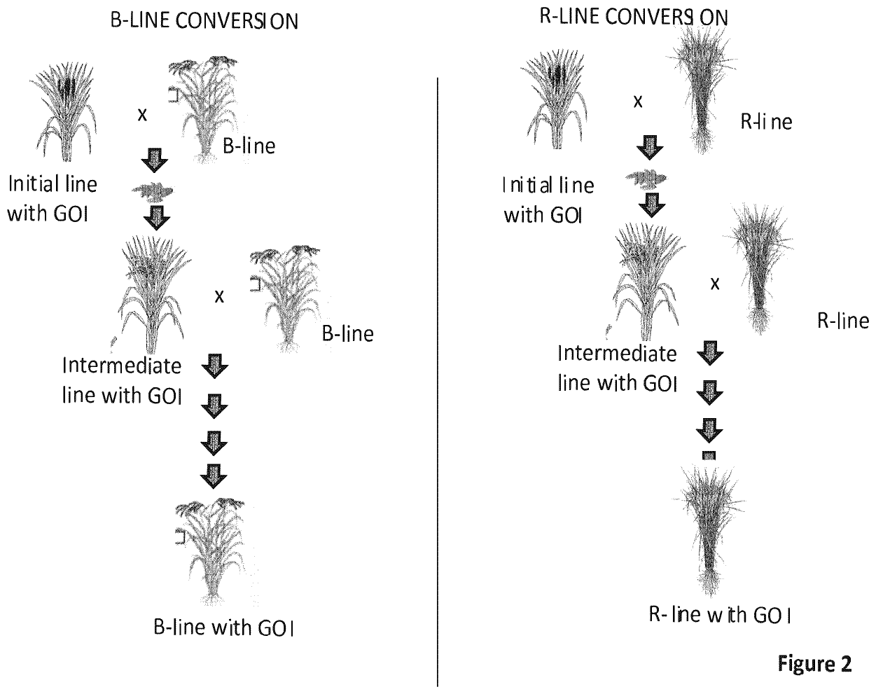

Since the male fertile lines such as pollinator or restorer lines as well as maintainer lines are both female and male fertile, conversion of such lines into lines further containing the gene(s) or allele(s) of interest can be conveniently achieved by repeated backcrossing with restorer lines or maintainer lines (see schematic representation in FIG. 2).

There is no need to fully convert the A-line to an A-line comprising the gene(s) or allele(s) of interest, which requires more time and is labor-intensive due to the male sterility of such line. Moreover, commitment to include a particular trait into the hybrids is only made at the last pre-basic seed stage and increases the versatility of commercial hybrid seed production to meet anticipated demand of hybrid seed containing a specific trait or combination of traits.

Thus, in a first embodiment of the invention, a method is provided for producing hybrid seed comprising a trait of interest using
a) a male-sterile plant line or A-line;
b) a male-sterile plant line comprising said trait of interest in heterozygous or hemizygous state or AGOI-line;
c) an isogenic maintainer plant line or B-line;
d) an isogenic maintainer plant line comprising said trait of interest in homozygous state or BGOI-line;
e) and a male fertile line, such as a pollinator line or restorer line or R-line comprising said trait of interest in homozygous state;
wherein said hybrid seed is produced by crossing of plants grown from basic seed of the AGOI-line and plants of the male fertile line or R-line and collecting the seeds produced on plants of the AGOI-line, and
wherein said basic seed of the AGOI-line has been produced by crossing plants grown from pre-basic seed of the A-line with plants grown from seed of the BGOI-line and collecting the seeds produced on plants of the A-line, and
wherein said pre-basic seed of the A-line has been produced by crossing plants grown from pre-basic seed of the A-line with plants grown from seed of the B-line and collecting seeds produced on plants of the A-line.

In a conventional three-line hybrid seed production system there are three different distinct genetic lines required for hybrid seed production. These include:
A-line (Cytoplasmic Male Sterile—infertile pollen and “sterile” cytoplasm)
B-line (Maintainer line—fertile pollen and “normal” cytoplasm)
Male fertile or pollinator line with fertile pollen which may comprise nuclear genes to over-ride the sterility mechanism such as that conferred by the cytoplasm. In the latter case the pollinator line is usually referred to as the R-line (Restorer line)

It should be noted that there is no need for restorer genes in case the harvested plant product is not the seed. Furthermore, in case of recessive (nuclear) male sterile genes any wt gene restoring the function of the male sterility gene may act as a restorer gene, and any male fertile line can act as “restorer” line. Furthermore, other systems are available to provide sufficient pollinator plants in the population of plants grown from the hybrid seed such as by blending unrestored hybrids made by CMS based production systems with hybrid seed made by non-CMS based production systems (e.g. detasseling in the case of corn).

Suitable parent lines for a three-line hybrid system can be identified and/or developed in the following manner. Initially, an “exotic” or wild progenitor may be required as the cytoplasm donor. This exotic germplasm can initially be used as a female and unknown lines can be crossed to this female to evaluate if sterility is “perfect”. Perfect sterility is found when the resulting F1 of this cross is sterile at maturity. Prior to heading, another check can be performed whereby florets are sampled, anthers are removed, anthers are squashed and pollen is stained with 5% iodine solution. Iodine staining is used to ascertain the starch content of the pollen grains. If pollen grains are devoid of starch (non-staining) at a level of 99.99% then the unknown line can be further considered as a possible maintainer which could lead to development of an A-line. The successive crosses with the unknown and the sterile off-spring should be sterile. The elite B-line can be transferred to a sterile cytoplasm. A perfect sterile A-line and a perfect maintainer or B-line can be obtained. The A-line and B-line are actually isolines differing only in cytoplasmic backgrounds.

If the unknown line produces very high levels of fertility (>85%) in the F1, then the unknown line would be considered a possible restorer. The same pollen staining prior to heading is utilized. If starch grains stained at greater than 95% then the unknown line is considered a possible restorer. Testing the F1 over numerous environments and obtaining the same results of high fertility would lead to the conclusion that this unknown line is a “perfect” restorer.

Three line hybrid seed production systems have been developed for several plant species:
A widespread triphyletic hybridization system for oilseed rape (Brassica napus) employs the Ogura cytoplasmatic sterility and fertility restoration (R40 or the improved R2000) from radish as described in WO92/05251, WO97/0737 or WO2005/002324.
In rice, most triphyletic hybridization system employ cytoplasmic male sterility identified in Wild abortive (WA) male sterile rice (Li, 1977, Acta Botanica Sinica 19:7-10) although diversified male sterile cytoplasms were developed in the 1980s.
In wheat, moderately successful commercialization of hybrids has been achieved, using the Triticum timopheevi CMS system (Wilson and Ross, 1962 Wheat Info. Serv. 14: 29-30).
Most of the hybrid sunflower production is based on a single source of cytoplasmic male sterility from Helianthus petiolaris (CMS) PET1, although alternative CMS sources and corresponding nuclear restorer lines have been described (Chepurnaya et al. 2003 HELIA 26 nr 38, pp59-66).
CMS systems have also been identified and characterized in many other crop species, including Phaseolus vulgaris, beet, carrot, maize, onion, petunia, rye and sorghum (Kück and Wricke 1995 Advances in Plant Breeding 18, Genetic Mechanisms for Hybrid Breeding Blackwell Wissenschafts-Verlag).

In addition to this hybrid seed production systems based on cytoplasmic sterility, there are other hybrid seed production systems which require a female, male and maintainer line. This is the case e.g. in the nuclear male sterility system based on expression of lethal gene to cause male sterility including anther specific expression of RNAses (such as barnase) and corresponding genes to “reverse” the effect of the expression of the lethal gene (including expression of an RNAse inhibitor such as barnase), referred to as SeedLink® or InVigor® described e.g. in WO 89/10396 and WO91/02069. Other transgene based hybrid seed production system including those based on pollen specific expression of lethal genes (such as described in WO93/25695) or based on selection of male sterile lines through visual markers (such as described in WO 95/34634). Another hybrid seed production system is referred to as Seed Production Technology® (Pioneer Hi-bred) as described e.g. in WO 2009/103049, US2009/0038026 or US2006/0288440. It will be immediately clear that the methods and schemes described herein may also be applied to these hybrid seed production systems.

Tryphyletic hybrid seed production systems are widespread and are used commercially to produce hybrid seeds for rice, millet, sorghum, canola, cabbage, corn etc.

To increase the volume of seed to plant the next generation and to produce more A-line crossing of A×B lines is required. The maintainer line (inbred in normal or fertile cytoplasm) is used as a pollen source to maintain the female (inbred with sterile cytoplasm). The seed produced from this out-crossing is again sterile as the nucleus of the maintainer cannot over-ride the sterility mechanism conferred by the sterile cytoplasm. This A×B seed production maintains the female sterile line and increases the amount of female seed.

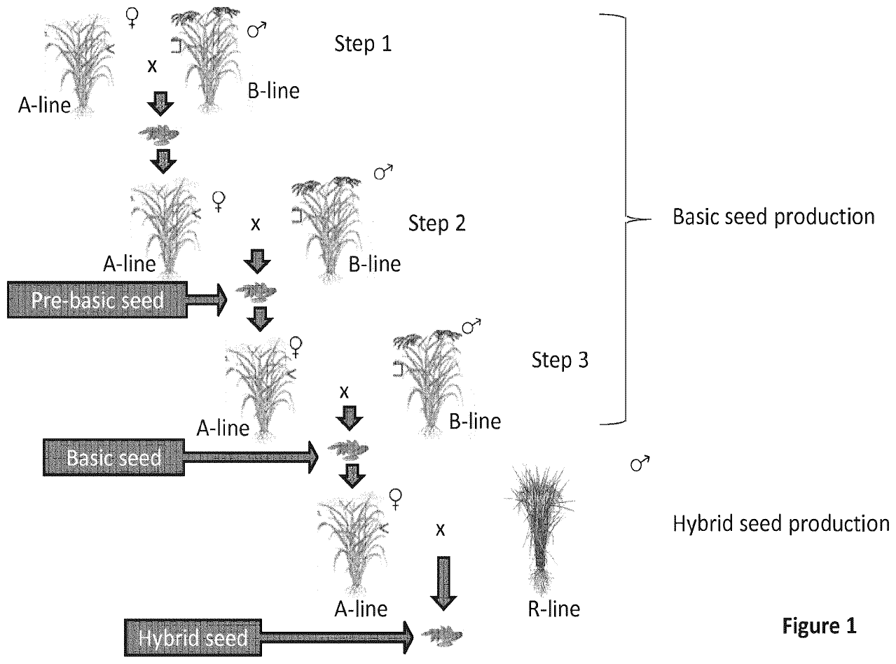

Several generations of maintenance breeding are required to build up enough female seed to utilize in hybrid seed commercial production (A×R) acres. A typical stepwise increase or production scheme would include (see also FIG. 1):
1^{st} Step: seed is produced by hand crossing A and B lines to increase to several thousand female seeds. Isolation in bags is done to ensure genetic integrity of the seed.
2^{nd} Step: The seed of the A-line produced in step 1 is planted in small acreages (percentage of hectare to several hectares) together with B-line pollinator plants. A×B increase rows or strips are pollinated by utilizing wind, insects, or other methods of supplemental pollination to increase female seed to hundreds of kilograms of seed. Isolation from other pollen sources is required to ensure genetic integrity of the female seed. The A-line seed collected from this stage is often referred to as “pre-basic seed”.
3^{rd} Step: A×B seed is planted out in larger acreages (10 s to 100 s of hectare). A×B grown to produce thousands of kilograms of female seed. Isolation is required to ensure the genetic integrity of the female seed. This 3^{rd} step concludes the female increase. This female seed is planted as female stripes in the A×R seed production fields in the hybrid seed production stage. The A-line seed collected from this stage is referred to as “basic seed” or “foundation seed”.

Hybrid seed production requires crossing of the A-line×a male fertile line such as an R-line to produce F1 seed. The restorer line (inbred which may contain nuclear gene(s) to over-ride cytoplasm) is used as a pollen source to outcross with the A-line to produce F1 seed. The seed produced from this out-crossing can be fertile as the nucleus of the pollinator line may over-ride the sterility mechanism when it is considered a restorer line.

In a particular embodiment of the invention, the trait of interest is herbicide tolerance and the gene(s) or allele(s) are herbicide tolerance conferring genes or alleles. The invention thus also provides a method for producing herbicide-tolerant hybrid seed using
a) a male-sterile plant line (A-line);
b) a male-sterile plant line comprising a herbicide tolerance gene in heterozygous or hemizygous state (AHT-line);
c) an isogenic maintainer plant line (B-line);
d) an isogenic maintainer plant line comprising a herbicide tolerance gene in homozygous state (BHT-line);
e) and a male fertile line which may be a restorer line, comprising said herbicide tolerance gene in homozygous state (R-line);
said method comprising the steps of
i. producing pre-basic seed of the A-line by crossing plants grown from seed of the A-line with plants grown from seed of the B-line and collecting seeds produced on plants of the A-line;
ii. producing basic seed of the AHT-line by crossing plants growing from pre-basic seed of the A-line with plants growing from seed of the BHT-line and collecting the seeds produced on plants of the A-line; and
iii. producing hybrid seed by crossing plants grown from basic seed of the AHT-line and plants of the male fertile line, particularly the R-line and collecting the seeds produced on plants of the AHT-line.

In other words, the invention provides an improvement to a method for producing herbicide tolerant hybrid seeds using a three-line hybridization system comprising a male-sterile plant line or A-line, a isogenic maintainer plant line or B-line and a restorer liner or R-line comprising a herbicide tolerance gene, wherein the improvement comprises crossing a fourth isogenic maintainer line further comprising a herbicide tolerance gene in homozygous state with the male-sterile plant only at the stage of basic seed production and not at any pre-basic seed production stages.

The BHT-line can be obtained by crossing a plant of a B-line with an HT gene donor (preferably a dominant HT gene, preferably in homozygous form) and performing back-crosses with the non-HT B-line as recurrent parent. Backcrossing would take 3 to 6 generations depending on whether backcrossing with molecular markers to add conversion or conventional backcrossing would be utilized for the gene transfer. Each BHT-line reaching 100% conversion can then be increased. Since the B-line is self-pollinating this process is fast and efficient. The BHT-line is a third isogeneic-line to the A-line and the B-line.

In Steps 1 and 2 of the hybrid seed production scheme as represented in FIG. 3, the A-line and B-line are non-herbicide tolerant lines. The herbicide tolerant B-line is used in Step 3 of the seed increase scheme for the A×B production. The A-line seed produced in this method will be hemizygous for the herbicide tolerance gene(s) or allele(s) (AHT-line). This hemizygous AHT-line (foundation seed) is used to plant the female strips in the A×R hybrid seed production fields. An herbicide tolerant R-line is used as the restorer in the hybrid seed production fields. The herbicide tolerant F1 or hybrid seed produced in this method will be ½ hemizygous and ½ homozygous for the herbicide tolerance gene(s) or allele(s). All herbicide tolerant hybrid seed planted in the next generation in the hybrid fields will survive the herbicide application.

A first advantage of the herein described hybrid seed production system is that all non-herbicide tolerant plants can be killed by application of the appropriate herbicide in the hybrid seed production fields and therefore increase the purity of the hybrid seeds produced. Non-herbicide tolerant plants could originate form:
a. B-line seed mixed into the A-line increases of Step 1 and Step 2.
b. Genetic variants mixed into the A-line increase from volunteers in the field.
c. Errant pollen out-crossing onto the A-line.
d. Ad-mixture into the A-line seed from any sources throughout the process.
e. Partial male fertile female plants present in Step 1 and Step 2 of A-line seed production due to environmental conditions reducing the 99.99% sterility levels required to maintain the A-line.

These forms of genetic variants are usually removed from the field by hand through a process described as rogueing whereby any genetic variants are removed from both the A-line and R-line strips prior to flowering in the A×R hybrid seed production. Additional rogueing is also done on the A-line strips prior to harvest of the A×R hybrid seed production fields. This rogueing is costly and labor intensive. Rogueing is also a subjective process performed by many individuals walking large acreages. Each individual may have a different view of what constitutes a variant to be removed. Moreover, each individual checking a designated area may not consistently remove perceived variants at 100% at all times and in all locations.

Removal of genetic variants by application of herbicides is much more efficient and effective and leads to a significant reduction in cost and complexity of hybrid seed production.

A further advantage of the seed production systems herein described is the ability to utilize the same female for any number of herbicide tolerance type systems. If the female in the start of Step 1 is tolerant or resistant to one herbicide it is dedicated and can only be utilized for that herbicide tolerance system. The system described herein makes the commitment to a particular herbicide tolerance only in the 3rd stage of basic seed production.

Additional benefits include:
No need for multiple A-lines each with a different HT technology.
A perfect female and maintainer are required to produce more A-line seed. Development of a new A-line requires 3 to 4 backcrosses with the new respective B-line. Each step requires pollen exam and review of bagged panicles (in cereals) to ensure the seed from A line x B-line increase is completely sterile.
Lines used as donors of the HT gene may not be perfect maintainers. Genes linked or close to the site of the HT gene on the chromosome may cause partial fertility. Due to the closeness of the genes or linkage, it may be very difficult if not impossible to develop a new perfect female with 99.99% fertility. Partial fertility in the HT A-line in Step 3 of the A-line increase could be acceptable if the F1 seed produced in the A×R hybrid seed fields meet seed purity standards.
Time to market is not prevented or delayed because of the A-line conversion.
The number of partially fertile or fully fertile genetic variants in a three step A-line increase will increase in each step of production. Acceptable purity standards can be harder to reach if out-crossed seed produced in Step 1 and Step 2 is lower relative to selfed seed of the partial of fully fertile genetic variants.

A further advantage of the herein described seed production system is the ease of development and increase of herbicide tolerant B-lines as this will come from separate HT B-line increases. The amount of HT B-line produced can be based upon the market demand for each HT hybrid system. However, the volume of A-line seed produced in Step 1 and Step 2 of A-line seed production can be used as the female in Step 3 of seed production since it is the B-line which determines the nature of the A-line seed produced in the Step 3. At Step 3, the A-line could comprise any type of herbicide tolerance or even be non-herbicide tolerant. Market demand for specific herbicide tolerances varies over time due to evolving weed spectrum. The methods provided herein are easier and require less time to adapt to a changing market demand for different types of herbicide tolerance. The methods also require a less complex seed inventory system.

Herbicide tolerance may be provided by a transgene(s) or by (variant) endogenous allele(s).

It will be immediately apparent that the exact nature of the herbicide tolerance gene(s) or allele(s) is not critical to the seed production system, although preferably it is a herbicide tolerance gene or allele providing commercial resistance when present in hemizygous form in a plant, and preferably is a dominant herbicide tolerance gene.

Suitable herbicide tolerance genes may provide tolerance against a herbicide from the class of
acetyl CoA carboxylase inhibitors (such as aryloxyphenoxypropionates including fenoxaprop, fluazifop or quizalafop; cyclohexanediones including clethodim or sethoxydim)
acetolactoate synthase inhibitors (such as sufonylureas including chlorimuron, foramsulfuron, halosulfuron, iodosulfuron, nicosulfuron, primisulfuron, prosulfuron, rimsulfuron, thifensulfuron or tribenuron; imidazolinones including imazamox, imazaquin or imazethapyr; tryazolopyrimidines including flumetsulam or cloransulam; triazolinones including thiencarbazone)
glutamine synthetase inhibitors (including glufosinate)
5-enoylpyruvyl-shikimate-3-phosphate inhibitors (including glyphosate)
photosynthesis II inhibitors (such as triazines including atrazine or simazine; triazinones including metribuzin; nitriles including bromoxynil; benzothiadazoles including bentazon; ureas including linuron)
diterpene synthesis inhibitors (including isoxazolidinone)
hydroxyphenylpyruvate dioxygenase inhibitors (isoxazoles including isoxaflutole; pyrzolones including topramezone; triketones including mesotrione and tembotrione)
protoporphorinogen oxidase inhibitors (diphenylethers including acifluorfen, formesafen, lactofen; N-phenylphtalimides including flumiclorac or flumioxazin; aryl triazinone including sulfentrazone, carfentrazone or fluthiacet-ethyl; pyrimidinediones including saflufenacil)
photosystem I electron diverters (such as bipyridilium including paraquat)
microtubule inhibitors (such as dinitroanilines including ethalfluralin, pendimethalin or trifluralin)
lipid synthesis inhibitors (such as thiocarbamate including butylate or EPTC or S-ethyl-N,N-dipropylthiocarbamate)
long chain fatty acid inhibitors (such as chloroacetamides including acetochlor, alachlor, metolachlor, dimethenamid; oxyacetamides including flufenacet; pyrazoles including pyroxasulfone)
synthetic auxins (such as phenoxys including 2,4-D; benzoic acids including dicamba; carboxylic acid including clopyralid or fluroxypyr; semicarbazones including difluofenzopyr).

The following herbicide tolerance genes or alleles or events may be suitable for the hybrid seed production schemes described herein:
Glufosinate tolerance genes, such as the bar gene or the pat gene as described e.g. in WO8705629 or U.S. Pat. No. 5,276,268 or the DSM-2 gene described in WO2009152359. Rice plants containing such glufosinate tolerance genes include rice plants containing Event LLRICE06 (Rice, herbicide tolerance) deposited as ATCC-23353, described in WO2000/026356, or described in regulatory reference US98-329-01p; Event LLRICE601 (Rice, herbicide tolerance) deposited as ATCC PTA-2600, described in US20082289060, or described in regulatory reference US06-234-01p; Event LLRICE62 (Rice, herbicide tolerance) deposited as ATCC-203352, described in WO2000/026345, or described in regulatory reference US98-329-01p. Oilseed rape plants containing a glufosinate tolerance gene include OSR plants containing Event RF3 (Oilseed Rape, pollination control and herbicide tolerance) deposited as ATCC PTA-730, described in WO2001/041558, or described in regulatory reference US98-278-01p. Sugar beet plants containing a glufosinate tolerance gene include sugar beet comprising Event T-120-7 (Sugarbeet, herbicide tolerance) described in regulatory reference US97-336-01p.
Glyphosate tolerance genes, such as 2mepsps described in e.g. WO9704103 or cp4 described in e.g. WO92/04449. Rice plants comprising glyphosate tolerance genes include rice plants comprising Event 17053 (Rice, herbicide tolerance) deposited as ATCC PTA-9843, described in WO2010/117737 or Event 17314 (Rice, herbicide tolerance) deposited as ATCC PTA-9844, described in WO2010/117735. Wheat plants comprising glyphosate tolerance genes include wheat plants comprising event Event 33391 (Wheat, herbicide tolerance) deposited as PTA-2347, described in WO2002027004. Oilseed rape plants comprising glyphosate tolerance genes include OSR plants comprising events Event MON88302 (Oilseed Rape, herbicide tolerance) deposited as, described in WO2011/153186, or described in regulatory reference US11-188-01p or Event RT73 (Oilseed Rape, herbicide tolerance) not deposited, described in WO2002/036831, or described in regulatory reference US98-216-01p.
Imidazoline tolerance alleles as described e.g. in WO2004106529 (wheat), WO2004040011 (OSR), WO2009135254 (barley), WO2005/020673 (rice) EP1659855 (rice).
HPPD inhibitor tolerance genes such as those described e.g. in WO 2011/076892, WO2011/076889, WO 2011076885, WO 2011076882, WO2011076877, WO2011068567, WO2010085705 or WO2009144079.
2,4-D tolerance genes such as those described in e.g. WO88/01641, WO2005/107437, WO 2007053482 or WO2008141154.
Dicamba tolerance genes such as those described e.g. in WO2007146678.
Variant Acetyl-coenzyme A carboxylase encoding alleles tolerant to ACCase inhibiting herbicides, particularly FOPS, are described e.g. in WO2013/016210 (rice) or WO2012/106321(wheat) amongst others.

The following is a list of transgenic events, containing gene(s) of interest conferring herbicide tolerance, stress tolerance, insect control, disease tolereance, quality or yield traits, or combinations thereof which could be introduced into hybrid plants using the methods described herein: Event J101 (Alfalfa, herbicide tolerance) described in regulatory reference US04-110-01p; Event J163 (Alfalfa, herbicide tolerance) described in regulatory reference US04-110-01p; Event KK179-2 (Alfalfa, quality trait) deposited as ATCC PTA-11833, described in WO2013/003558; Event ASR-368 (Bent grass, herbicide tolerance) deposited as ATCC PTA-4816, described in WO2004053062, or described in regulatory reference US03-104-01p; Event EE-1 (Brinjal, insect control) not deposited, described in WO2007/091277; Event RM3-3 (Chicory, pollination control and herbicide tolerance) described in regulatory reference US97-148-01p; Event RM3-4 (Chicory, pollination control and herbicide tolerance) described in regulatory reference US97-148-01p; Event RM3-6 (Chicory, pollination control and herbicide tolerance) described in regulatory reference US97-148-01p; Event 32316 (Corn, insect control and herbicide tolerance) deposited as ATCC PTA-11507, described in WO2011/084632; Event 3272 (Corn, quality trait) deposited as ATCC PTA-9972, described in WO2006098952, or described in regulatory reference US05-280-01p; Event 40416 (Corn, insect control and herbicide tolerance) deposited as ATCC PTA-11508, described in WO2011/075593; Event 4114 (Corn, insect control and herbicide tolerance) deposited as ATCC PTA-11506, described in WO2011/084621; Event 43A47 (Corn, insect control and herbicide tolerance) deposited as ATCC PTA-11509, described in WO2011/075595; Event 5307 (Corn, insect control) deposited as ATCC PTA-9561, described in WO2010/077816; Event 676 (Corn, pollination control and herbicide tolerance) not deposited, described in regulatory reference US97-342-01p; Event 678 (Corn, pollination control and herbicide tolerance) not deposited, described in regulatory reference US97-342-01p; Event 680 (Corn, pollination control and herbicide tolerance) not deposited, described in regulatory reference US97-342-01p; Event B16 (Corn, herbicide tolerance) deposited as ATCC 203059, described in US2003126634, or described in regulatory reference US95-145-01p; Event BT11 (Corn, insect control and herbicide tolerance) described in regulatory reference US95-195-01p; Event BT176 (Corn, insect control and herbicide tolerance) described in regulatory reference US94-319-01p; Event CBH351 (Corn, insect control and herbicide tolerance) described in regulatory reference US97-265-01p; Event DAS40278 (Corn, herbicide tolerance) deposited as ATCC PTA-10244, described in WO2011/022469, or described in regulatory reference US09-233-01p; Event DAS-59122-7 (Corn, insect control and herbicide tolerance) deposited as ATCC PTA-11384, described in WO2006/039376, or described in regulatory reference US03-353-01p; Event DAS-59132 (Corn, insect control and herbicide tolerance) not deposited, described in WO2009/100188; Event DBT418 (Corn, insect control and herbicide tolerance) deposited as, or described in regulatory reference US96-291-01p; Event DP-098140-6 (Corn, herbicide tolerance) deposited as ATCC PTA-8296, described in WO2008/112019, or described in regulatory reference US07-152-01p; Event DP-32138-1 (Corn, hybridization system) deposited as ATCC PTA-9158, described in WO2009/103049, or described in regulatory reference US08-338-01p; Event FI117 (Corn, herbicide tolerance) deposited as ATCC 209031, described in WO1998/044140; Event GA21 (Corn, herbicide tolerance) deposited as ATCC 209033, described in WO1998/044140, or described in regulatory reference US97-099-01p; Event GG25 (Corn, herbicide tolerance) deposited as ATCC 209032, described in WO1998/044140; Event GJ11 (Corn, herbicide tolerance) deposited as ATCC 209030, described in WO1998/044140; Event HCEM485 (Corn, herbicide tolerance) described in regulatory reference US09-063-01p; Event LY038 (Corn, quality trait) deposited as ATCC PTA-5623, described in WO2005061720, or described in regulatory reference US04-229-01p; Event MIR162 (Corn, insect control) deposited as ATCC PTA-8166, described in WO2007/142840, or described in regulatory reference US07-253-O1p; Event MIR604 (Corn, insect control, not deposited, described in WO2005103301, or described in regulatory reference US04-362-01p; Event MON80100 (Corn, insect control and herbicide tolerance) described in regulatory reference US95-093-01p; Event MON802 (Corn, insect control and herbicide tolerance) described in regulatory reference US96-317-01p; Event MON809 (Corn, insect control and herbicide tolerance) described in regulatory reference US96-017-01p; Event MON810 (Corn, insect control, not deposited, described in US2004180373, or described in regulatory reference US96-017-01p; Event MON863 (Corn, insect control) deposited as ATCC PTA-2605, described in WO2004/011601, or described in regulatory reference US01-137-01p; Event MON87427 (Corn, pollination control) deposited as ATCC PTA-7899, described in WO2011/062904, or described in regulatory reference US10-281-01p; Event MON87460 (Corn, stress tolerance) deposited as ATCC PTA-8910, described in WO2009/111263, or described in regulatory reference US09-055-O1p; Event MON88017 (Corn, insect control and herbicide tolerance) deposited as ATCC PTA-5582, described in WO2005/059103, or described in regulatory reference US04-125-01p; Event MON89034 (Corn, insect control) deposited as ATCC PTA-7455, described in WO2007/140256, or described in regulatory reference US06-298-O1p; Event MS3 (Corn, pollination control and herbicide tolerance) deposited as, or described in regulatory reference US95-228-01p; Event MS6 (Corn, pollination control and herbicide tolerance) described in regulatory reference US95-228-01p; Event MZDT09Y (Corn, stress tolerance) deposited as ATCC PTA-13025, described in WO2013/012775; Event NK603 (Corn, herbicide tolerance) deposited as ATCC PTA-2478, described in US2007-056056, or described in regulatory reference US97-099-01p; Event T14 (Corn, herbicide tolerance) described in regulatory reference US94-357-14p; Event T25 (Corn, herbicide tolerance) not deposited, described in WO2001/051654, or described in regulatory reference US94-357-01p; Event TC1507 (Corn, insect control and herbicide tolerance) not deposited, described in WO2004/099447, or described in regulatory reference US00-136-0p; Event TC6275 (Corn, insect control and herbicide tolerance) described in regulatory reference US00-136-O1p; Event VIP1034 (Corn, insect control and herbicide tolerance) deposited as ATCC PTA-3925., described in WO2003/052073; Event 1076 (Cotton, insect control, not deposited, described in regulatory reference US94-308-01p; Event 1143-14A (Cotton, insect control, not deposited, described in WO2006/128569; Event 1143-51B (Cotton, insect control, not deposited, described in WO2006/128570; Event 1445 (Cotton, herbicide tolerance) not deposited, described in WO2002/034946, or described in regulatory reference US95-045-01p; Event 1698 (Cotton, herbicide tolerance) deposited as, or described in regulatory reference US95-045-01p; Event 19-51A (Cotton, herbicide tolerance) not deposited, described in regulatory reference US95-256-O1p; Event 281-24-236 (Cotton, insect control and herbicide tolerance) deposited as ATCC PTA-6233, described in WO2005/103266, or described in regulatory reference; CA; DD2005-52; Event 3006-210-23 (Cotton, insect control and herbicide tolerance) deposited as ATCC PTA-6233, described in WO2005/103266, or described in regulatory reference US03-036-02p; Event 31807 (Cotton, insect control and herbicide tolerance) not deposited, described in regulatory reference US97-013-01p; Event 31808 (Cotton, insect control and herbicide tolerance) not deposited, described in regulatory reference US97-013-01p; Event 757 (Cotton, insect control, not deposited, described in regulatory reference US94-308-01p; Event BXN (Cotton, herbicide tolerance) described in regulatory reference US93-196-01p; Event CE43-67B (Cotton, insect control) deposited as DSM ACC2724, described in WO2006/128573, or described in regulatory reference US07-108-01p; Event CE44-69D (Cotton, insect control, not deposited, described in WO2006/128571; Event CE46-02A (Cotton, insect control, not deposited, described in WO2006/128572; Event COT102 (Cotton, insect control, not deposited, described in WO2004039986, or described in regulatory reference US03-155-01p; Event COT202 (Cotton, insect control, not deposited, described in WO2005054479; Event COT203 (Cotton, insect control, not deposited, described in WO2005/054480; Event GHB119 (Cotton, insect control and herbicide tolerance) deposited as ATCC PTA-8398, described in WO2008/151780, or described in regulatory reference US08-340-01p; Event GHB614 (Cotton, herbicide tolerance) deposited as ATCC PTA-6878, described in WO2007/017186, or described in regulatory reference US06-332-01p; Event LLcotton25 (Cotton, herbicide tolerance) deposited as ATCC PTA-3343, described in WO2003013224, or described in regulatory reference US02-042-01p; Event MON1076 (Cotton, insect control) described in regulatory reference US94-308-01p; Event MON15985 (Cotton, insect control) deposited as ATCC PTA-2516, described in WO2002/100163, or described in regulatory reference US00-342-01p; Event MON88701 (Cotton, herbicide tolerance) deposited as, Event MON88913 (Cotton, herbicide tolerance) deposited as ATCC PTA-4854, described in WO2004/072235, or described in regulatory reference US04-086-01p; Event T304-40 (Cotton, insect control and herbicide tolerance) deposited as ATCC PTA-8171, described in WO2008/122406; Event T342-142 (Cotton, insect control, not deposited, described in WO2006/128568; Event ARB-FTE1-08 (Eucalyptus, quality trait) deposited as, or described in regulatory reference US08-366-01p; Event CDC TRIFFID (Flax, herbicide tolerance) described in regulatory reference US98-335-01p; Event 23-18-17 (Oilseed Rape, quality trait, not deposited, described in regulatory reference US94-090-01p; Event 61061 (Oilseed Rape, herbicide tolerance) deposited as, Event HCN10 (Oilseed Rape, herbicide tolerance) described in regulatory reference US01-206-02p; Event HCN92 (Oilseed Rape, herbicide tolerance) described in regulatory reference US01-206-02p; Event MON88302 (Oilseed Rape, herbicide tolerance) deposited as, described in WO2011/153186, or described in regulatory reference US11-188-01p; Event MS11 (Oilseed Rape, pollination control and herbicide tolerance) deposited as ATCC PTA-850 or PTA-2485, described in WO2001/031042; Event MS8 (Oilseed Rape, pollination control and herbicide tolerance) deposited as ATCC PTA-730, described in WO2001/041558, or described in regulatory reference US98-278-01p; Event MS8 (Oilseed Rape, pollination control and herbicide tolerance) deposited as ATCC PTA-730, described in US2001029620, or described in regulatory reference; CA; DD96-17; Event OXY235 (Oilseed Rape, herbicide tolerance) described in regulatory reference; CA; DD98-25; Event RF1 (Oilseed Rape, pollination control and herbicide tolerance) deposited as, or described in regulatory reference US98-278-01p; Event RF2 (Oilseed Rape, pollination control and herbicide tolerance) described in regulatory reference US98-27-01p; Event RF3 (Oilseed Rape, pollination control and herbicide tolerance) deposited as ATCC PTA-730, described in WO2001/041558, or described in regulatory reference US98-278-01p; Event RT200 (Oilseed Rape, herbicide tolerance) described in regulatory reference US98-216-01p; Event RT73 (Oilseed Rape, herbicide tolerance) not deposited, described in WO2002/036831, or described in regulatory reference US98-216-01p; Event T45 (Oilseed Rape, herbicide tolerance) described in regulatory reference US97-205-01p; Event 55-1 (Papaya, virus resistance, not deposited, described in regulatory reference US96-051-01p; Event 63-1 (Papaya, virus resistance, not deposited, described in regulatory reference US96-051-01p; Event X17-2 (Papaya, virus resistance) described in regulatory reference US04-337-01p; Event N70 (Peanut, disease tolerance) deposited as, or described in regulatory reference US10-070-01p; Event P39 (Peanut, disease tolerance) deposited as, or described in regulatory reference US10-070-01p; Event W171 (Peanut, disease tolerance) described in regulatory reference US10-070-01p; Event C5 (Plum, disease tolerance) described in regulatory reference US04-264-01p; Event ATBT04-27 (Potato, insect control) described in regulatory reference US95-338-01p; Event ATBT04-30 (Potato, insect control) described in regulatory reference US95-338-01p; Event ATBT04-31 (Potato, insect control) described in regulatory reference US95-338-01p; Event ATBT04-36 (Potato, insect control) described in regulatory reference US95-338-01p; Event ATBT04-6 (Potato, insect control) described in regulatory reference US95-338-01p; Event BT10 (Potato, insect control) described in regulatory reference US94-257-01p; Event BT12 (Potato, insect control) described in regulatory reference US94-257-01p; Event BT16 (Potato, insect control) described in regulatory reference US94-257-01p; Event BT17 (Potato, insect control) described in regulatory reference US94-257-01p; Event BT18 (Potato, insect control) described in regulatory reference US94-257-01p; Event BT23 (Potato, insect control) described in regulatory reference US94-257-01p; Event BT6 (Potato, insect control) described in regulatory reference US94-257-01p; Event RBMT15-101 (Potato, insect control and virus tolerance) described in regulatory reference US97-339-01p; Event RBMT21-129 (Potato, insect control and virus tolerance) described in regulatory reference US97-204-01p; Event RBMT21-350 (Potato, insect control and virus tolerance) described in regulatory reference US97-204-01p; Event RBTM122-82 (Potato, insect control and virus tolerance—HERBICIDE TOLE) described in regulatory reference US97-204-0p; Event SBT02-5 (Potato, insect control) described in regulatory reference US95-388-01p; Event SBT02-7 (Potato, insect control) described in regulatory reference US95-338-01p; Event SEMT15-02 (Potato, insect control and virus tolerance) described in regulatory reference US97-339-0p; Event SEMT15-15 (Potato, insect control and virus tolerance) described in regulatory reference US97-339-0p; Event 17053 (Rice, herbicide tolerance) deposited as ATCC PTA-9843, described in WO2010/117737; Event 17314 (Rice, herbicide tolerance) deposited as ATCC PTA-9844, described in WO2010/117735; Event LLRICE06 (Rice, herbicide tolerance) deposited as ATCC-23353, described in WO2000/026356, or described in regulatory reference US98-329-01p; Event LLRICE601 (Rice, herbicide tolerance) deposited as ATCC PTA-2600, described in US20082289060, or described in regulatory reference US06-234-01p; Event LLRICE62 (Rice, herbicide tolerance) deposited as ATCC-203352, described in WO2000/026345, or described in regulatory reference US98-329-01p; Event PE-7 (Rice, insect control, not deposited, described in WO2008/114282; Event IFD-52401-4 (Rose, quality trait) described in regulatory reference US08-315-01p; Event IFD-52901-9 (Rose, quality trait described in regulatory reference US08-315-01p; Event 40-3-2 (Soybean, herbicide tolerance) described in regulatory reference US93-258-01p; Event BPS-CV127-9 (Soybean, herbicide tolerance) deposited as NCIMB No. 41603, described in WO2010/080829, or described in regulatory reference US09-015-01p; Event DAS21606 (Soybean, herbicide tolerance) deposited as ATTC PTA-11028, described in WO2012/033794; Event DAS44406 (Soybean, herbicide tolerance) deposited as PTA-11336, described in WO2012/075426; Event DAS68416 (Soybean, herbicide tolerance) deposited as ATCC PTA-10442, described in WO2011/066360, or described in regulatory reference US09-349-01p; Event DP-305423-1 (Soybean, quality trait, not deposited, described in WO2008/054747, or described in regulatory reference US06-354-01p; Event DP-356043-5 (Soybean, herbicide tolerance) deposited as ATCC PTA-8287, described in WO2008/002872, or described in regulatory reference US06-271-01p; Event FG72 (Soybean, herbicide tolerance) deposited as NCIMB 41659, described in WO2011063411, or described in regulatory reference US09-328-01p; Event G-168 (Soybean, quality trait) described in regulatory reference US97-008-01p; Event G94-1 (Soybean, quality trait) described in regulatory reference US97-008-01p; Event G94-19 (Soybean, quality trait) described in regulatory reference US97-008-01p; Event GU262 (Soybean, herbicide tolerance) described in regulatory reference US98-238-01p; Event LL27 (Soybean, herbicide tolerance) deposited as NCIMB41658, described in WO2006/108674, or described in regulatory reference US96-068-01p; Event LL55 (Soybean, herbicide tolerance) deposited as NCIMB 41660, described in WO2006/108675, or described in regulatory reference US96-068-01p; Event MON 87712 (Soybean) deposited as ATCC Accession No PTA-10296, described in WO2012/051199A2; Event MON87701 (Soybean, insect control) deposited as ATCC PTA-8194, described in WO2009/064652, or described in regulatory reference US09-082-01p; Event MON87705 (Soybean, quality trait and herbicide tolerance) deposited as ATCC PTA-9241, described in WO2010/037016, or described in regulatory reference US09-201-01p; Event MON87708 (Soybean, herbicide tolerance) deposited as ATCC PTA9670, described in WO2011/034704, or described in regulatory reference US10-188-01p; Event MON87712 (Soybean output trait) deposited as PTA-10296, described in WO2012/051199, or described in regulatory reference US11-202-01p; Event MON87754 (Soybean, quality trait) deposited as ATCC PTA-9385, described in WO2010/024976; Event MON87769 (Soybean, quality trait) deposited as ATCC PTA-8911, described in WO2009/102873, or described in regulatory reference US09-183-01; Event MON89788 (Soybean, herbicide tolerance) deposited as ATCC PTA-6708, described in WO2006/130436, or described in regulatory reference US06-178-01p; Event pDAB8264.42.32.1 (Soybean, herbicide tolerance) deposited as ATCC PTA-11993, described in WO2013/010094; Event pDAB8264.44.06.1 (Soybean, herbicide tolerance) deposited as ATCC Accession No PTA-11336, described in WO2012/075426A1, or described in regulatory reference US11-234-01p; Event pDAB8291.45.36.2 (Soybean;) deposited as ATCC Accession No PTA-11355, described in WO2012/075426; Event W62 (Soybean, herbicide tolerance) described in regulatory reference US96-068-01p; Event CZW-3 (Squash, virus resistance) described in regulatory reference US95-352-01p; Event ZW-20 (Squash, virus resistance) described in regulatory reference US92-204-01p; Event GM RZ13 (Sugarbeet, virus resistance) deposited as NCIMB-41601, described in WO2010/076212; Event GTSB77 (Sugarbeet, herbicide tolerance) described in regulatory reference US98-173-01p; Event H7-1 (Sugarbeet, herbicide tolerance) deposited as NCIMB 41158 or NCIMB 41159, described in WO2004/074492, or described in regulatory reference US03-323-01p; Event T-120-7 (Sugarbeet, herbicide tolerance) described in regulatory reference US97-336-01p; Event T227-1 (Sugarbeet, herbicide tolerance) not deposited, described in WO2002/44407; Event 21-41 (Tobacco, quality trait, not deposited, or described in regulatory reference US01-121-01p; Event 1345-4 (Tomato, quality trait) described in regulatory reference US94-228-01p; Event 35 1 N (Tomato, quality trait) described in regulatory reference US95-324-01p; Event 5345 (Tomato, insect control, not deposited, described in regulatory reference US97-287-01p; Event 8338 (Tomato, quality trait, not deposited, described in regulatory reference US95-053-01p; Event B (Tomato, quality trait) described in regulatory reference US94-209-01p; Event F (Tomato, quality trait) described in regulatory reference US94-290-01p; Event FLAVRSAVR (Tomato, quality trait) described in regulatory reference US94-227-01p; Event NT73 1436-111 (Tomato, quality trait) described in regulatory reference US96-248-01p; Event 33391 (Wheat, herbicide tolerance) deposited as PTA-2347, described in WO2002027004; Event JOPLIN1 (Wheat, disease tolerance) not deposited, described in US2008064032; Event MON71800 (Wheat, herbicide tolerance).

The methods described herein are not limited to use in particular plant species, but may be used in any plant species for which triphyletic hybrid seed production scheme are available, including rice (Oryza sativa), wheat (Triticum aestivum), corn (Zea mays), cotton (Gossypium hirsutum or G. barbadense), soybean (Glycine max), sorghum (Sorghum bicolor), rapeseed (Brassica napus), mustard seed (Brassica juncea), barley (Hordeum vulgare), oat (Avena sativa), rye (Secale cereale), pearl millet (Pennisetum typhoides), alfalfa (Medicago sativa), tomato (Lycopersicon esculentum), sugar beet (Beta vulgaris), sunflower (Helianthus annuus), onion (Allium cepa), petunia (Petunia hybrida) or carrot (Daucus carota) amongst others.

### Examples

Development of a hybrid seed production system based on herbicide tolerant mutant rice line HT1 resistant to herbicide H1:

### A. Development of Female A-Line:

1) Establish “elite” A-line (UA3) and elite B-line (UB3)
2) Confirm Perfect Female status by
   a. Pollen staining and exam
   b. Fertility under bagged plants
3) Obtain HT1-line 1 as herbicide tolerance (or resistance) donor
   a. Validate herbicide resistance or tolerance
   b. Select resistant plants
   c. Identify background of HT1-line 1
4) Develop B-line with HT1 resistance
   a. Cross UB3×HT1-line
      i. Spray HT1-F1 seedlings with herbicide (H1)
      ii. Isolate resistant plants
   b. Cross UB3×HT1-F1
      i. Spray HT1-BC1 seedlings with herbicide (H1)
      ii. Isolate resistant plants
      iii. Use markers to identify plants with highest % of UB3 genome
   c. Cross UB3×HT1-BC1
      i. Spray with HT1-BC2 seedlings with herbicide (H1)
      ii. Isolate resistant plants
      iii. Use markers to identify plants with highest % of UB3 genome
   d. Cross UB3×HT1-BC2
      i. Spray with HT1-BC3 seedlings with herbicide (H1)
      ii. Isolate resistant plants
      iii. Use markers to identify plants with highest % of UB3 genome
5) Select the lines of HT1-UB3 which are the best isogenic lines of UB3
6) Make confirmation test cross (CTC) with isolines
   a. F1 UA3×UB3 (standard or check)
   b. F1 UA3×isolines HT1-UB3
7) Compare and confirm Perfect Maintainer
   a. Pollen staining and exam of CTC-F1
   b. Fertility under bagged CTC-F1
8) Continue conversion backcrossing (CBC) and continue female validation
   a. BC1: F1CTC×HT1-UB3
      i. Pollen staining
      ii. Fertility under bagged plants
   b. BC2: CBC1×HT1-UB3
      i. Pollen staining
      ii. Fertility under bagged plants
   c. BC3: CBC2×HT1-UB3
      i. Pollen staining
      ii. Fertility under bagged plants
   d. Derived converted plants consisting of HT1-UA3 and HT1-UB3
9) Establish lines HT1-UA3 and HT1-UB3
10) Produce Pre-Basic Seed
   a. UA3 and UB3
      i. UA3×UB3
      ii. UB3 panicle rows
   b. HT1-UA3×HT1-UB3
      i. HT1-UA3×HT1-UB3
      ii. HT1-UB3 panicle rows
11) Produce Basic Seed of Female
   a. UA3×UB3
   b. UA3×HT1-UB3
   c. HT1-UA3×HT1-UB3
12) Identify the following Production data
   a. Flowering dates
   b. Flowering traits
   c. Compare volumes of seed produced by each method
13) Keep Basic Seed for hybrid seed production
   a. UA3
   b. F1 HT1-UA3
   c. HT1-UA3

### B. Development of Male Line:

1. Establish “elite” R-line UR007
2. Confirm Perfect Male through Heterosis test cross (HetTC)
   a. Pollen staining and exam of F1 (>95% fertilite pollen)
   b. Fertility of bagged F1 plants (>85% seed set)
   c. Agronomic data on F1 including vigor, flowering, height, and yield
3. Produce UR007 Male
4. Obtain HT1-line 1 as herbicide tolerance (or resistance) donor
   a. Validate herbicide resistance or tolerance
   b. Select resistant plants
   c. Identify background of HT1-line 1
5. Develop R-line with HT1 resistance
   a. UR007×HT1-line 1
      i. Spray HT1-F1 seedling herbicide (H1)
      ii. Isolate resistant plants
   b. UR007×HT1-F1
      i. Spray HT-BC1 seedling with herbicide (H1)
      ii. Isolate resistant plants
      iii. Use markers to identify plants with highest % of UR007 genome
   c. UR007×HT1-BC1
      i. Spray HT1-BC2 seedlings with herbicide (H1)
      ii. Isolate resistant plants
      iii. Use markers to identify plants with highest % of UR007 genome
   d. UR007×HT1-BC2
      i. Spray HT1-BC3 seedlings with herbicide (H1)
      ii. Isolate resistant plants
      iii. Use markers to identify plants with highest % of UR007 genome
6. Select best lines of HT1-UR007 as isoline(s) of UR007
7. Make heterosis test cross (HetTC) with isolines
   a. F1 UA3×UR007 (standard or check)
   b. F1 UA3×isolines HT1-UR007
8. Compare and confirm Perfect Restorer UR007 AND HT1-UR007
   a. Pollen staining and exam of F1 (>95% fertilite pollen)
   b. Fertility of bagged F1 plants (>85% seed set)
   c. Agronomic data on F1 including vigor, flowering, height, and yield
9. Establish lines HT1-UR007
10. Produce Pre-Basic Seed of
   a. UR007
   b. HT1-UR007
11. Produce Basic Seed of
   a. UR007
   b. HT1-UR007
12. Collect the following Production data
   a. Flowering dates
   b. Flowering traits
   c. Compare volumes of seed produced by each method
13. Keep Basic Seed for hybrid seed production of
   a. UR007
   b. HT1-UR007

### C. Experimental Seed Production:

1. Experimental Seed Production
   a. Plant Basic Seed into ESP block of UA3×UR007 (stage A.13.a and B.13.a)
   b. Plant Basic Seed into ESP of UA3×HT1-UR007 (stage A.13.a and B.13.b)
   c. Plant Basic Seed ESP of F1 HT1-UA3×HT1-UR007 (stage A.13.b and B.13.b)
d. Plant Basic Seed ESP of HT1-UA3×HT1-UR007 (stage A.13.c and B.13.b)
2. Review Seed Production Agronomics of four ESP blocks
   a. Spray H1 on F1-HT1-UA3 and homozygous-HT1-UA3×HT1-UR007 blocks
   b. Utilize alternative herbicide on UA3×UR007 and UA3×HT1-UR007 blocks
3. Determine the following characteristics
   a. Flowering of A and R lines
   b. Flowering characteristics
   c. GA response
   d. Flowering
   e. Seed Set
   f. Total yield
4. Analyze Seed Production Techniques
   a. no difference in seed yield—use F1 HT1-UA3×HT1-UR007 method
5. Analyze purity of hybrid seed
   a. presence of weed seeds
   b. presence of red rice seeds
   c. presence and origin of genetic variants
      i volunteers
      ii B-line
      iii R-line
Seed production according to the methods of the invention result in equal quantity and purity as conventional systems.

### D. F1 Hybrid Testing:

1. Plant and compare agronomics of three H1 tolerant F1 hybrids
   a. Emergence
   b. Spray H1 and review symptomology on F1 “types”
   c. Determine vigor
   d. Determine flowering date
   e. Determine Height
   f Determine Lodging
   g. Determine Heterosis
   h. Determine Harvest characteristics
   i. Determine Yield
   j. Determine Milling (head and total yields)
   k. Determine Quality (amylose and gel point)
   l. Determine Taste
2. Plant checks in sidebar next to H1 sprayed Yield Trial
   a. F1 UA3/UR007
   b. Other leading checks or hybrids
   c. Review agronomic characteristics and performance
3. Analyze purity of hybrid seed plots
   Seed production according to the methods of the invention result in equal quantity and purity as conventional systems.

### E. Pre-commercial Seed Production:

1. Pre-commercial Seed Production Blocks
   a. Plant Basic Seed of UA3×HT1-UR007 (stage A.13.a and B.13.b)
   b. Plant Basic Seed of F1 HT1-UA3×HT1-UR007 (stage A.13.b and B.13.b)
2. Review Seed Production Agronomics of two pre-commercial seed production blocks
   a. Spray H1 on F1-HT1-UA3/HT1-UR007 block
   b. Utilize alternative herbicide F1-UA3/HT1-UR007 block
   c. Determine Flowering of A and R lines
   d. Determine Flowering characteristics
   e. Determine GA response
   f Determine Seed Set
   g. Determine Total yield
3. Analyze purity of hybrid seed
   a. presence of weed seeds
   b. presence of red rice seeds
   c. presence and origin of genetic variants
      i volunteers
      ii B-line
      iii R-line
         Seed production according to the methods of the invention result in equal quantity and purity as conventional systems.

### F. Pre-Commercial F1 Hybrid Testing:

1. Plant and compare agronomics of two H1 tolerant F1 hybrids
   a. Emergence
   b. Spray H1 and review symptomology on F1 “types”
   c. Determine Vigor
   d. Determine Flowering date
   e. Determine Height
   f Determine Lodging
   g. Determine Heterosis
   h. Determine Harvest characteristics
   i. Determine Yield
   j. Determine Milling (head and total yields)
   k. Determine Quality (amylose and gel point)
   l. Determine Taste
2. Analyze purity of F1 hybrid fields
   Seed production according to the methods of the invention result in equal quantity and purity as conventional systems.

### G. Acceptance of New Production Method:

1. Improve method of seed production so H1 can be used in Basic Seed Production
2. Improve Basic Seed production yields as H1 improves weed and red rice control
3. Maintain hybrid yields using new method of basic seed production
4. Improve levels of genetic purity in F1 seed and ensure no red rice in F1 seed
5. Eliminate steps A.6 through A.9 whereby maintainer is converted to a perfect female
6. Produce ‘standard’ Pre-basic UA3 seed
7. Produce Pre-basic HT1-UB3 seed and/or other HT2 or HT3 materials
8. Produce Basic F1 HT1-UA3 seed
9. Produce Basic seed of HT1-UR007 and/or other HT2 or HT3 materials
10. Produce Hybrid Seed using F1 HT1-UA3 as female and HT1-UR007 as restorer

1. A method for producing herbicide-tolerant hybrid seed using
(a) a male-sterile plant line (A-line);
(b) a male-sterile plant line comprising an herbicide tolerance gene in heterozygous or hemizygous state (AHT-line);
(c) an isogenic maintainer plant line (B-line);
(d) an isogenic maintainer plant line comprising an herbicide tolerance gene in homozygous state (BHT-line);
(e) and a male fertile plant line comprising said herbicide tolerance gene in homozygous state (R-line);
said method comprising the steps of
i) producing pre-basic seed of the A-line by crossing plants grown from pre-basic seed of the A-line with plants of the B-line and collecting seeds produced on plants of the A-line, wherein the method does not introduce herbicide tolerance at any pre-basic seed production stages of the A-line;
ii) producing basic seed of the AHT-line by crossing plants grown from said pre-basic seed of the A-line with plants of the BHT-line and collecting the seeds produced on plants of the A-line; and
iii) producing hybrid seed by crossing plants grown from basic seed of the AHT-line and plants of the R-line and collecting the seeds produced on plants of the AHT-line.

2. The method of claim 1, wherein said herbicide tolerance gene provides tolerance against a herbicide selected from the group of acetyl CoA carboxylase inhibitors, acetolactate synthase inhibitors, glutamine synthetase inhibitors, 5-enolpyruvyl-shikimate-3-phosphate synthase inhibitors, photosynthesis II inhibitors, diterpene synthesis inhibitors, hydroxyphenylpyruvate dioxygenase inhibitors, protoporphyrinogen oxidase inhibitors, photosystem I electron diverters, microtubule inhibitors, lipid synthesis inhibitors, long chain fatty acid inhibitors or synthetic auxins.

3. The method according to claim 3, wherein said herbicide tolerance gene provides tolerance against a glutamine synthase inhibitor herbicide and said herbicide is glufosinate.

4. The method of claim 1, wherein the male fertile line is a restorer line.

5. The method of claim 1, wherein said herbicide tolerance is provided by a transgene.

6. The method of claim 4, wherein said herbicide tolerance is provided by a transgene.

7. The method of claim 1, wherein said herbicide tolerance is provided by a variant allele endogenous to said plant.

8. The method of claim 4, wherein said herbicide tolerance is provided by a variant allele endogenous to said plant.
